(19) United States
(12) Patent Application Publication
(10) Pub. No.: US 2008/0086156 A1
(43) Pub. Date: Apr. 10, 2008

(54) METHODS AND DEVICES FOR USING DRUG-ELUTING EMBOLIZATION

(76) Inventors: Robert Rosen, New York, NY (US); Nicholas Kipshidze, New York, NY (US); Sriram Iyer, New York, NY (US); Gary Roubin, New York, NY (US)

Correspondence Address: MAYER BROWN LLP, P.O. BOX 2828, CHICAGO, IL, 60690, US

(21) Appl. No.: 11/745,203

(22) Filed: May 7, 2007

Related U.S. Application Data
(63) Continuation of application No. 11/532,034, filed on Sep. 14, 2006, now abandoned.
(60) Provisional application No. 60/717,125, filed on Sep. 14, 2005.

(51) Int. Cl. A61B 17/03

(52) U.S. Cl. 606/158

(57) ABSTRACT

A method and system of embolizing an organ or vessel is disclosed. An embolization device such as a coil, balloon, spheres, stents or occlusive plaques is used. The embolization device has a surface, which may be coated with composition retaining material such as a polymer, bio-polymer or non-polymer based technology that allows sustained release of drug or material from drug eluting occlusive device. For example, an anti-angiogenic composition is coated on the application surface. The embolization device is then inserted in the vessel and embolization is enhanced by the composition.

### FIELD OF INVENTION

This invention relates to devices relating to therapeutic embolization to occlude or modify blood flow and specifically to devices which are coated with either pro-thrombotic, angiogenic, anti-angiogenic, anti-neoplastic drugs, living cells or combinations thereof to achieve a sustained release and therefore highly specific local effect.

### BACKGROUND OF INVENTION

Currently, therapeutic intravascular embolization has become an essential tool for interventional medicine. Embolization is a method of occluding (closing) one or more blood vessels that need to be closed for various reasons such as hemorrhage, feeding the growth of a tumor or an arteriovenous malformation (AVM), an abnormal communication between an artery and a vein. The term “embolization” derives from embolus, which can be any object that circulates in the bloodstream until it lodges in a blood vessel.

Transcatheter occlusion devices have been used for treatment of many pathologies including patent ductus arteriosus (PDA), aortopulmonary collateral vessels, hemorrhages, aorto-pulmonary shunts, arteriovenous malformations, organ ablation, renal arteriovenous fistulas, coronary artery fistulas, and intracranial aneurysm occlusion. Therapeutic intravascular embolization has also been successfully used to devascularize neoplasms.

Several broad categories of vascular occlusion strategies can be recognized: liquid embolic agents (such as cyanoacrylate), sclerosing agents (such as ethanol, sodium tetradecyl sulfate), particulate agents (polyvinyl alcohol sponge particles), microspheres, biodegradable pledgets (gelatin sponge, collagen), and mechanical intravascular embolization devices (coils, umbrellas, plugs, detachable balloons).

Liquid embolic agents offer the advantages of low viscosity for easy injection through small catheters or catheters with many bends through tortuous blood vessels. During sclerotherapy, a chemical solution is injected into the blood vessel and the sclerosing agent irritates the walls of the vessel and clots the blood, causing permanent thrombosis. Cyanoacrylate adhesives rapidly polymerize after intravascular injection, forming a cast of the vessel with permanent occlusion—this almost instantaneous occlusion is useful in treating high flow lesions.

Polyvinyl alcohol embolization particles and other microsphere agents are artificial embolization devices used to obstruct or reduce the blood flow to hypervascular or neoplastic lesions via superselective catheter delivery.

A gelatin sponge generally behaves as a temporary agent in many vascular beds. Metallic coils and detachable balloons are frequently used to occlude larger vessels (arteries and veins). Detachable balloons are made of latex, silicone or other conforming materials. These balloons are delivered on a catheter and inflated with either isotonic contrast or a slowly polymerizing liquid plastic. However, detachable balloons are not ideal embolic materials because of their cost and complex delivery systems.

Major considerations for choosing an embolic occluding agent are speed and reliability of delivery, duration of occlusive effect, and preservation of normal tissue. The most widely used permanent vascular occlusion device has long been coil devices, which includes microcoils and macrocoils. Macrocoils, also called Gianturco coils have undergone several modifications to improve thrombogenicity and delivery systems. Coils have the advantage of precise positioning via fluoroscopic control. Embolization occurs as a result of coil-induced thrombosis as well as mechanical occlusion of the lumen by the coil. To increase the thrombogenic effect, Dacron fibers may be attached to coils, providing controlled delivery with rapid occlusion. These coils are available in a wide variety of sizes and may be delivered through commonly used angiographic catheters.

However, embolization with coils requires placement of a catheter at the targeting site. This is not always possible as a result of small vessel size or tortuous blood vessels, i.e. vessels with repeated twists and bends. Moreover, embolization coils have serious limitations, including early and late recanalization, insufficient control and predictability to make them safe, incomplete occlusion, complicated and large delivery systems, geographic miss and migration (in some cases, even the optimal arrangement of the coil alone cannot prevent migration). Collateralization is a potential disadvantage of coil embolization resulting in the persistence of flow into the vascular territory of the vessel that was embolized. Additionally, when proximal occlusion occurs with coil embolization, repeat intervention via the same artery becomes difficult, if not impossible.

A complication of coil occlusion using a Cook embolization coil is migration of coils into peripheral vessels, especially in patients having relatively larger size arteries. Multiple coils are often required to occlude vessels of large diameters, theoretically increasing the potential risk of imprecise placement. The stability of the stainless-steel spring coils is greatly influenced by their size. When coils are too small for a given vessel, they tend to migrate and if they are too large, they tend to remain elongated. Recanalization after coil embolization is also a problem. The use of such purely mechanical devices for embolization are thus not optimal as the walls of the blood vessel may shift preventing the embolization.

Thus, there is a need for a method of embolization that provides more control of occlusion of blood flow through the embolized vessel. There is also a need for a device and method of embolization which prevents development of collateralization during the process. There is a further need for an embolization method which prevents recanalization of the occluded segment. Finally, there is a need for an “active” coil or device which incorporates the release of specific active pharmacologic agents which can either (1) inhibit angiogenesis, (2) promote angiogenesis in ischemic states, or (3) release chemotherapeutic or other antineoplastic agents in a controlled fashion into a specific part of the circulation.

### SUMMARY OF THE INVENTION

These needs and others may be met by the present invention of which one embodiment is an intravascular (intra-tubular) embolization device for occluding or modifying flow in blood vessels (artieries, veins), in the lymphatic system, pathologic flow or flow to diseased organ. This embodiment has a mechanical blocking device having an application surface, the mechanical blocking device being insertable within a blood vessel. A pharmaceutically active composition is coated on the application surface, and the composition is in contact with the blood vessel.

It is to be understood that both the foregoing general description and the following detailed description are not limiting but are intended to provide further explanation of the invention claimed. The accompanying drawings, which are incorporated in and constitute part of this specification, are included to illustrate and provide a further understanding of the method and system of the invention. Together with the description, the drawings serve to explain the principles of the invention.

### BRIEF DESCRIPTION OF DRAWINGS

These and further aspects and advantages of the invention will be discussed more in detail hereinafter with reference to the disclosure of preferred embodiments, and in particular with reference to the appended Figures wherein:

FIG. 1A is a cross section of an artery with an embolization coil according to the present invention; and

FIG. 1B is a cross section of an artery with an embolization coil deployed according to the present invention.

### DESCRIPTION OF THE PREFERRED EMBODIMENT

While the present invention is capable of embodiment in various forms, there is shown in the drawings and will hereinafter be described a presently preferred embodiment with the understanding that the present disclosure is to be considered as an exemplification of the invention, and is not intended to limit the invention to the specific embodiment illustrated.

One device for use with, for example, an artery is a vascular occlusion device (VOD). The occlusion device has a series of porous cylinders. In this example the cylinders each have a length of 1.5 cm and a diameter of 6.0 mm and are machined from a biodurable, reticulated, elastomeric, resilient, polyurethane matrix. In another example the cylinders collectively have a length of 1.5 cm and a diameter of 6.0 mm.

The vascular occlusion device is inserted into the vessel via a catheter. Preferably a loader device is used to assist with compression and insertion of the device into a delivery catheter. In this example, the loader is a plastic handle with a short stainless steel tube.

Before implantation, the cylinders are coated with a substance which enhances embolization in the vessel. The substance composition depends on the length of time the user wishes to embolize the vessel. In the case of trauma where rapid embolization is desired, the device is coated with a hemostatic reagent such as thrombin or other thrombogenic substances such as fibrin gel, acrylic glue or other glues, or other hemostatic solutions and agents, or suitable combinations.

In the case of an arteriovenous connection, an anti-angiogenic compound is used to prevent development of collateral circulation. Anti-angiogenic agents inhibit neovascularization and therefore blood flow. Anti-angiogenesis drugs are thus locally administered to the affected area via coating on the device 10. These drugs include bevacizumab (Avastin®), Vitaxin®, angiostatin, endostatins and others.

In order to assist in retaining the composition coating, the cylinders or stents or other occlusion devices may be coated with phosphorylcholine (PC), a naturally occurring biological substance. The biocompatible PC coating constitutes a 50-100 nm thick double layer of synthetic PC coating that is able to absorb a drug via a sponge-like mechanism. A preferred process of impregnating a PC-coated cylinder is as follows. The device is immersed into a solution or suspension of an anti-angiogenic agent such as bevacizumab (Avastin®), which was mixed according to the manufacturer's instructions (i.e., 25 mg/ml). The device is immersed for at least about 5 minutes. After removal of the device from the solution and allowing it to dry for at least about 1 minute, about 10 micro liters of the same drug solution is pipetted onto the device. The PC polymer on the cylinders acts like a sponge in absorbing the drug solution/suspension. The device 10 is again allowed to air dry for 1 minute. Then the above process is repeated.

After air-drying for about 5 minutes, the device may be immediately deployed into the patient's vessel with the catheter. About 0.01 to about 10.0 micrograms/mm² of the drug can be impregnated using this method. Any anti-angiogenic agent (e.g., Vitaxin®, bevacizumab, angiostatin, endostatin), or a combination thereof, can be employed in the above process. The amount of drug impregnated into the device may be varied depending on the location and nature of pathology.

In the case of cancer treatment such as isolating a tumor, chemotherapeutic drugs such as paclitaxel and its derivates, monoclonal antibodies, interleukin (IL), interferon (INF), rapamycin (macrolide antibiotic), everolimus and analogues may be used.

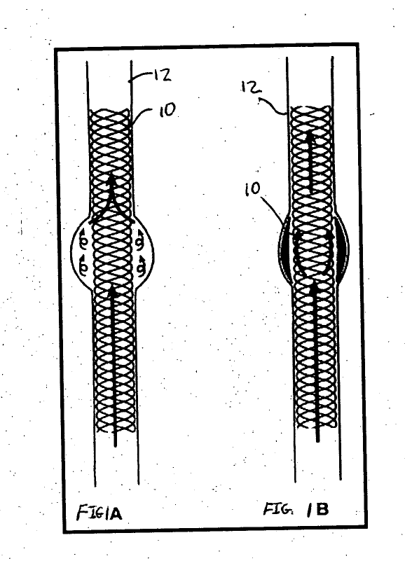

FIG. 1A shows the use of a stainless steel embolization coil 10 in a blood vessel 12 which requires embolization. The coil 10 may be of varying length, diameter, and loop configuration for different sized blood vessels. Coil embolization is a catheter-based procedure that allows precise occlusion of abnormal blood flow in a blood vessel. A catheter with the metallic occluding coil is inserted into an artery, usually in the groin (the femoral artery). It is then advanced to the abnormal blood vessel as shown in FIG. 1A. Once properly positioned, the metal coil 10 is released, springing into position within the vessel 12. It remains firmly in place by the expansion of the metal coils as shown in FIG. 1B. A blood clot will form on the coil, completely obstructing the abnormal blood flow beyond the coil. Eventually a scar will form, creating a permanent seal. In order to aid in the embolization process, the coil 10 is coated with a composition which aids clotting as explained above.

The use of compositions to aid embolization may be used with any mechanical embolization device. For example, the polyvinyl alcohol embolization particles are artificial embolization devices which may be coated. The particles are used to obstruct or reduce the blood flow to hypervascular or neoplastic lesions via superselective catheter delivery.

A detachable balloon to occlude larger vessels such as arteries and veins may preferably be coated with an anti-angiogenic composition. The detachable balloons are made of latex, silicone or other conforming materials. These balloons are delivered on a catheter and inflated with either isotonic contrast or a slowly polymerizing liquid plastic. The coating is then put in contact with the walls of the vessel aiding in embolization.

Another device which may use coatings to aid the embolization process is a polymer plaque. Different polymers such as non-erodable and non-biodegradable and bioderodable polymers and bio-polymers, such as fibrin, collagen, chitosan may be used. Another device is a metal stent which is coated with the compositions described above and inserted in the vessel to elute the drug to enhance the embolization process of the target vessel or organ.

It will be apparent to those skilled in the art that various modifications and variations can be made in the method and system of the present invention without departing from the spirit or scope of the invention. Thus, the present invention is not limited by the foregoing descriptions but is intended to cover all modifications and variations that come within the scope of the spirit of the invention and the claims that follow.

What is claimed is:

1. An embolization device for occluding flow in blood vessels, the device comprising:
a mechanical blocking device having an application surface, the mechanical blocking device being insertable within a blood vessel; and
a pharmaceutically active composition coated on the application surface, the composition in contact with the blood vessel.

2. The embolization device of claim 1 wherein the pharmaceutically active composition comprises an anti-angiogenic agent.

3. The embolization device of claim 2 wherein the mechanical blocking device comprises a series of cylinders.

4. The embolization device of claim 2 wherein the mechanical blocking device comprises a coil.

5. The embolization device of claim 2 wherein the mechanical blocking device comprises a stent.

6. The embolization device of claim 2 wherein the mechanical blocking device comprises a series of spheres.

7. The embolization device of claim 2 wherein the mechanical blocking device comprises a collapsible balloon.

8. The embolization device of claim 2 wherein the mechanical blocking device comprises a plaque.

9. The embolization device of claim 2 wherein the application surface comprises a phosphorylcholine coating.

10. The embolization device of claim 1 wherein the pharmaceutically active composition comprises an anti-neoplastic agent.

11. The embolization device of claim 10 wherein the anti-neoplastic agent is selected from the group consisting of paclitaxel and its derivatives, monoclonal antibodies, interleukin, interferon, rapamycin, everolimus, and analogues and combinations thereof.

12. The embolization device of claim 1 wherein the pharmaceutically active composition comprises a hemostatic agent.

13. The embolization device of claim 12 wherein the hemostatic agent is selected from the group consisting of thrombin, other thrombogenic substances, and combinations thereof.

14. The emobolization device of claim 13 wherein the other thrombogenic substances are selected from the group consisting of fibrin gel, acrylic glue, other glues, and combinations thereof.

15. The embolization device of claim 2 wherein the pharmaceutically active composition further comprises an anti-neoplastic agent, a hemostatic agent, or both.

16. A method of embolizing a blood vessel comprising:
coating an application surface of a blocking device with a composition selected from the group consisting of anti-angiogenic agents, anti-neoplastic agents, anti-hemostatic agents, and combinations thereof; and
inserting the blocking device in the blood vessel to embolize the vessel.

17. The method of claim 16 wherein the composition is an anti-angiogenic agent.

18. The method of claim 16 wherein the composition is an anti-neoplastic agent.

19. The method of claim 16 wherein the composition is an anti-hemostatic agent.

20. The method of claim 16 wherein the composition comprises at least two agents selected from the group consisting of anti-angiogenic agents, anti-neoplastic agents, anti-hemostatic agents, and combinations thereof.
